(19) United States
(12) Patent Application Publication
(10) Pub. No.: US 2012/0046517 A1
(43) Pub. Date: Feb. 23, 2012

(54) Second generation tape

(76) Inventor: Konstantin Lazarov Valtchev, Toronto (CA)

(21) Appl. No.: 12/806,629

(22) Filed: Aug. 18, 2010

(51) Int. Cl. A61F 2/02

(52) U.S. Cl. 600/30

(57) ABSTRACT

A method for treating female stress urinary incontinence by implantable tapes. One embodiment are woven and knitted tapes having only macropores, a macropore is constructed by single threads, the threads at the crossing areas are bonded by heating them to the melting point of the surface of the threads. Other embodiment is molded tapes, having only macropores, a macropore is constructed by single threads. The crossing crossing areas of the threads may have a simple crossing configuration, a spherical crossing configuration and a cubical crossing configuration. A molded tape having one or three sections which have different construction.

References Cited
1. Uster F C, Chaser J & Tuttle Jr. LLD. Use of Matrix mesh in the repair of incision Hernias. Am Surge 1958; 24:969.
2. Ulmsten U. & Petros P. Intravaginal slingoplasty (IVS) an ambulatory surgical procedure for treatment of female utinary incontinence. Scand J Urol Nephrol 1995; 29 (1):75-82.
3. Birch C. Best Practice & Research Clinical Obstetrics and Gynaecology Vol. 19, No 6, pp 979-991, 2005.
4. Milani R. Salvatore S. Soligo M. Pifarotti P. Mesvhia M. & Cortese M. Functional outcome of anterior and posterior vaginal prolapse repaire with prolene mesh. BJOG 2005; 112:107-111.
5. Cumberland V H. A preliminary report on the fabricated nylon weave in the repair of ventral hernias. Med J Aust 1952; 1:143-144.
6. Scales JT. Materials for hernia repair. Proc R. Sac Med 1953; 46:647-652.
   U.S. Patent Documents

| | | |
|---|---|---|
| 6,273,852 B1 | Aug. 2001 | Jorn Lehe |
| 6,638,211 B2 | Oct. 2003 | Patrice Suslian |
| 7,025,063 B2 | Apr. 2006 | Eva S. Snitkin |
| 7,393,320 B2 | Jul. 2008 | Karen Pilney Montpetit |
| 7,347,813 B2 | Mar. 2008 | Jan Claren |

### BACKGROUND OF THE INVENTION

The present invention relates generally to a synthetic mesh used for pelvic tape made of polypropylene or other synthetic material for treatment of stress urinary incontinence and vaginal prolapse.

Stress urinary incontinence (SUI) is a common symptom in elderly women, especially those who have multiple vaginal deliveries. It is an involuntary loss of urine when an intra-abdominal pressure is increased. The mechanisms for stress urinary incontinence are complex. Most classifications identify two main groups; urethral hypermobility and intrinsic sphincter deficiency. The object of incontinence surgery is to increase outlet obstruction, achieved by either repositioning the bladder neck to its correct anatomical position or to create a platform, which causes compression of the urethra during increase of the intra-abdominal pressure. Synthetic slings made of polypropylene, introduced as Marlex, was used in 1958¹. The tension free vaginal tape (TVT), the most successful synthetic sling procedure, was developed in 1990 by Ulmsten and Petros². Their tape is made from polypropylene. At the present time, on the market there are more than 10 different tapes and instruments for insertion of TVT and meshes for treatment of pelvic organ prolapse.

Clinical experiences during the past four decades with knitted and woven implants made from non-absorbable synthetic material has shown, that the rate infection, rejection and expulsion of the implants depends upon the size of the pores, the thickness of the thread, are the threads monofilament or multifilament, and the weight of the implant per sq. m. The pores size in excess of 75 nm (0.075μ) is considered as a macropore³ allowing fast infiltration of fibroblasts, new blood vessels formation of collagen fibres, which decrease infection rate. The multifilament thread can trap bacteria between the filaments leading to infection and rejection of the prosthesis. The monofilament thread has been proven to be less associated with infection. Some of the meshes are knitted with two threads running in one direction. Between the two threads however are formed micropores from 100 microns to less than 1 microns. These micropores can trap bacteria causing infection and possibly erosion. The meshes with lesser weight per sq. m have been shown to have a decreased rejection rate. Milani R. et al⁴ have reported an erosion rate of 13% in 63 women treated with polypropylene prosthesis for a mean of 17 months after the operation. The ideal prosthetic material should be biocompatible, inert, have minimal allergic or inflammatory reaction, non-carcinogenic, resistant to infection, withstanding mechanical stress at a reasonable cost^{5,6}. Until now we do not have a prosthetic material possessing all necessary requirements.

Accordingly, what is needed is a new improved tape, and device and method for implanting the same.

### SUMMARY OF THE INVENTION

The present invention provides an implant for use as a tape in the treatment of stress urinary incontinence and pelvic organ prolapse. A method and a sling introducer for installing a tape through a vaginal incision is described in details in my U.S. Pat. No. 7,338,432, which is given here as a reference. In this patent application, details of the surgical procedure describing the placement of the tape on both side of the urethra will be omitted for avoiding repetitions and focusing for clarity only on the present invention.

A method is provided for a tape made of woven or knitted mesh having only macropores and the threads are bonded together, at the area of crossing of the threads, using only one thread for construction of macropores, eliminating in this way formation of micropores. If two or more threads are used to construct a pore inevitably micropores will be formed. The threads are bonded together at the area of their crossings by heating them, for example by ultrasonic energy, to the melting point of the surface of the threads.

A method is also provided for a tape which is molded to desired length, width, size of the macropores, having as well different configuration of the areas of crossing of the threads. The areas of crossing of the threads can have a spherical, cubical, arrow like or simple crossing configuration. A method is also provided for a molded tape having similar construction from one end to the opposed end or the molded tape is divided in three or more sections and each section has different direction and shape of the threads, different configuration of the area of crossing of the threads. The threads can have different cross section, circular, square, hexagonal, rectangular and others. The goal is a tape free of micropores, having a good grip to the tissue, moderate elasticity and resistant to rolling. There are other features and advantages of the present invention which will become obvious from the following description of the drawings and detailed description of the invention.

### BRIEF DESCRIPTION OF THE DRAWING

FIG. 1a is a plan exploded view of a pore of woven tape.

FIG. 1b is a cross section of threads at their crossings.

FIG. 2a is a plan exploded view of a pore of a knitted tape.

FIG. 2b is a cross section of threads at their crossings.

FIG. 3a is a plan exploded view of a pore of a molded tape with a simple crossing configuration.

FIG. 3b is a cross section of the threads at their crossings.

FIG. 4a is a plan exploded view of a pore of a molded tape with a cubical crossing configuration.

FIG. 4b is a cross section of the threads at their crossings.

FIG. 5a is a plan exploded view of a pore of a molded tape with a spherical crossing configuration.

FIG. 5b is a cross section of the threads at their crossings.

FIG. 6a is a plan view of a molded tape having a simple crossing configuration and one of hypotenuse of macropores is aligned with longitudinal axis of the molded tape.

FIG. 6b is a plan view of a molded tape having three sections, a middle section, a first end section and a second end section, the middle section is outlined by a single thread.

FIG. 6c is a plan view of a molded tape with three sections, a middle section, a first end section and a second end section. The middle section has different orientation of macropores.

FIG. 7 is a plan view of other embodiment of a molded tape having three sections, a first and second end sections having a spherical crossing configuration and a middle section having simple crossing configuration.

### DETAILED DESCRIPTION

The present invention is intended primarily to be used as a tape for treating stress urinary incontinence, it is to be understood that the invention is not so limited, as there are other suitable applications such as pelvic reconstructive surgery or hernia repair.

The invention consists of two groups of implantable tapes made of polypropylene or other suitable plastic material. In a first group are woven and knitted tapes and in a second group are molded tapes. Both groups of tapes have only macropores.

Micropores are completely eliminated. In the first group that is achieved by bonding together the threads at their crossing areas heating them to the melting point of the surface of the threads. In the second group the same is achieved by molding the tapes. Macropores in both groups are constructed by single threads, eliminating formation of micropores.

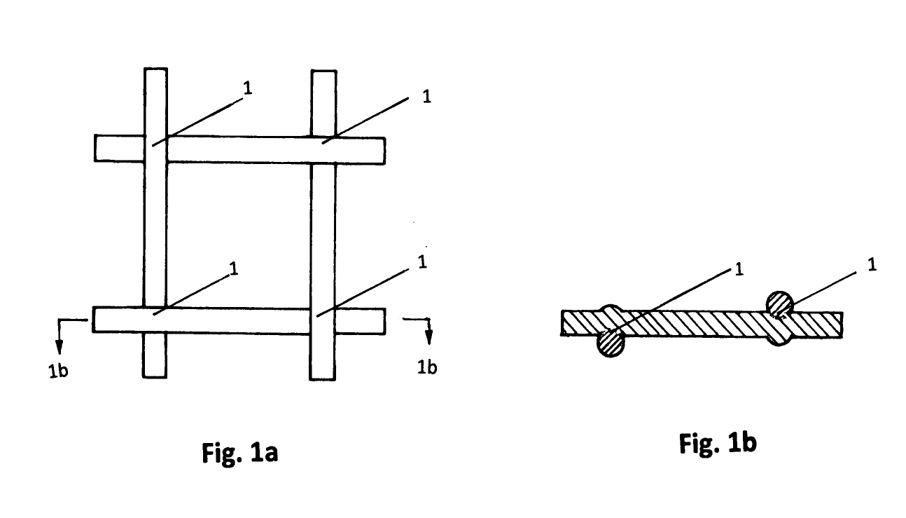

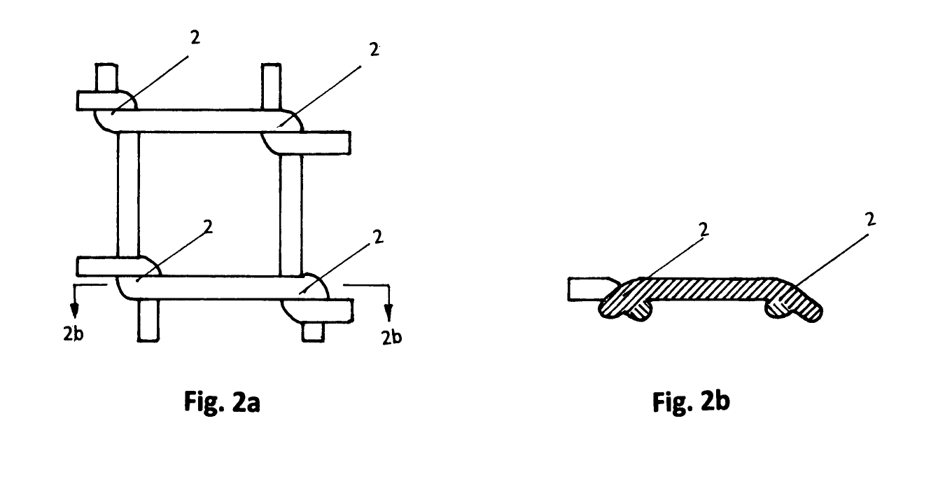

A macropore of the first group of tapes, woven and knitted, are shown on FIGS. 1a and 1b and on FIGS. 2a and 2b.

On FIGS. 1a and 1b is shown a macropore of a woven tape, made from polypropylene. The macropore is constructed by single threads. Threads are bonded together at the crossing areas 1, seen on the cross section on FIG. 1b. As was said above, by heating the surface of the threads to their melting point. Using only single threads eliminates formation of micropores. If two or more threads are used together, inevitably micropores are formed between them. Bonding the threads together at their crossings will eliminate micropores, which inevitably will form, if the threads are not bonded. Any micropores can harbour bacteria, which may lead to infection and possible rejection and expulsion of the tape.

On FIG. 2a is shown a macropore of a knitted tape made of polypropylene. The macropore is constructed by single threads. The threads are bonded together at the crossing areas 2, seen on the cross section on FIG. 2b, by heating the surface of the threads to their melting point. Dimensions of the tapes from the first group are similar to dimensions of the second group tapes.

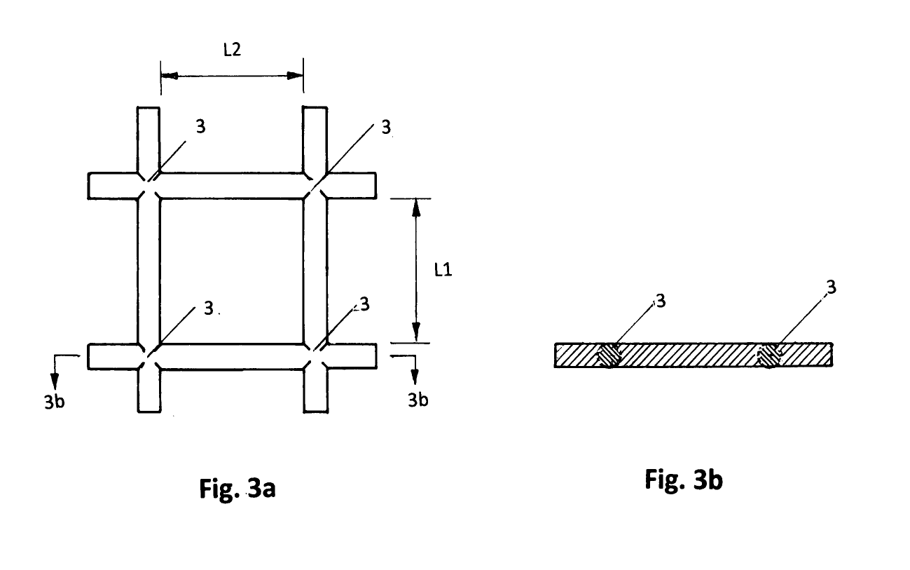

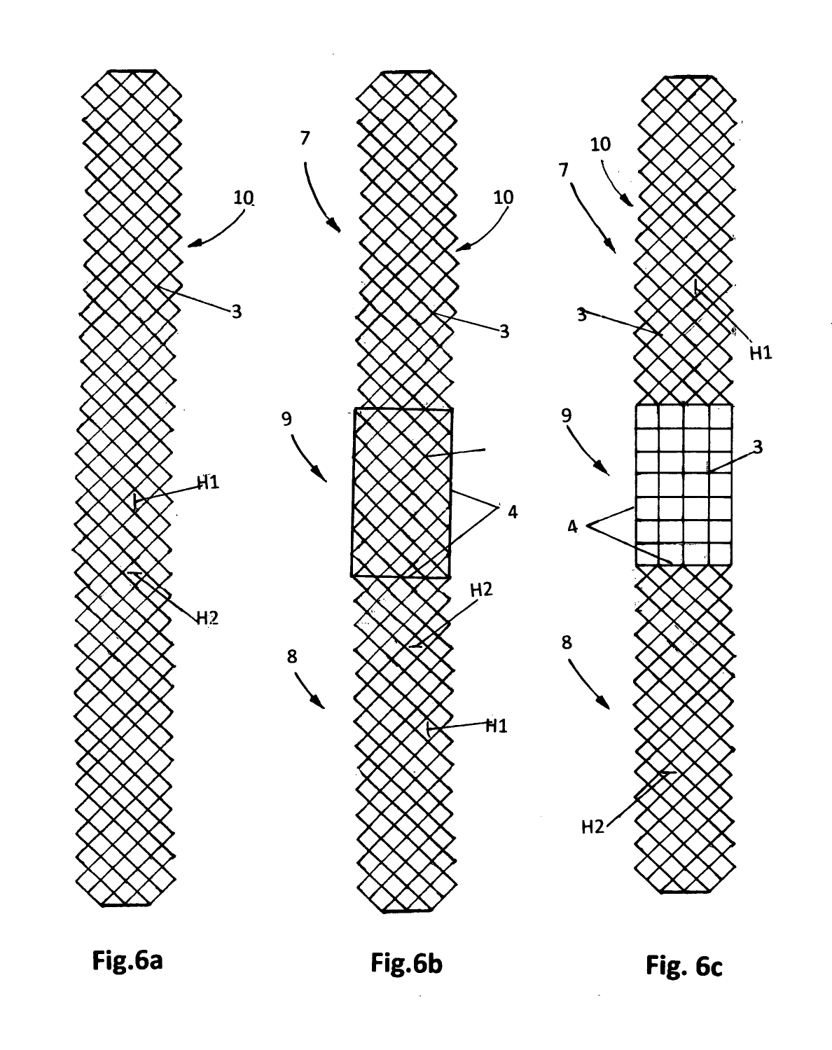

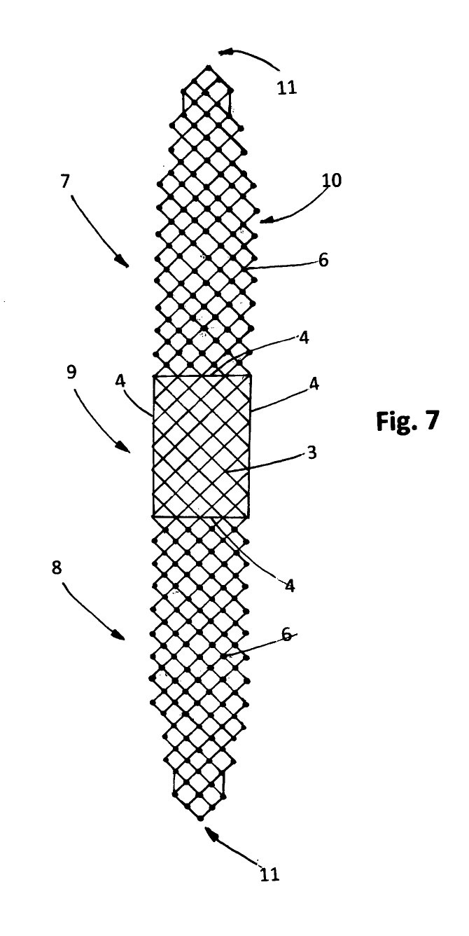

The second group tapes shown on FIGS. 3a and 3b, FIGS. 4a and 4b, FIGS. 5a and 5b, FIGS. 6a-6c and FIG. 7. The molded tapes may have more than one section. Going back to FIGS. 6 and 6c and FIG. 7 are shown molded tapes with three sections, a middle section 9, a first end section 7 and a second end section 8. Different sections may have different construction. The great advantage of the molded tapes is that they can be made in innumerable forms according to our specifications. The molded tapes are made of polypropylene or other suitable plastic material. The molded tapes have only macropores constructed by single threads. The crossing areas of the threads have different configurations. On FIG. 3a is shown a simple crossing configuration 3, cross section of the simple crossing configuration 3 is shown on FIG. 3b. The diameters of the threads of a simple crossing configuration 3 remain the same at the crossing areas. A tape with a simple crossing configuration 3 has a smooth surface, less friction during insertion into the tissue, is more pliable and is more stretchable. The simple crossing configuration 3 is more suitable for that part of the tape, which lies over the urethra. In order to reduce the stretching of the middle section 9, it is surrounded by additional straight threads 4, see FIGS. 6b and 6c and FIG. 7. The molded tapes shown on FIGS. 6a-6c have simple crossing configurations 3. Only the middle section of the molded tape shown on FIG. 7 have also simple crossing configurations 3.

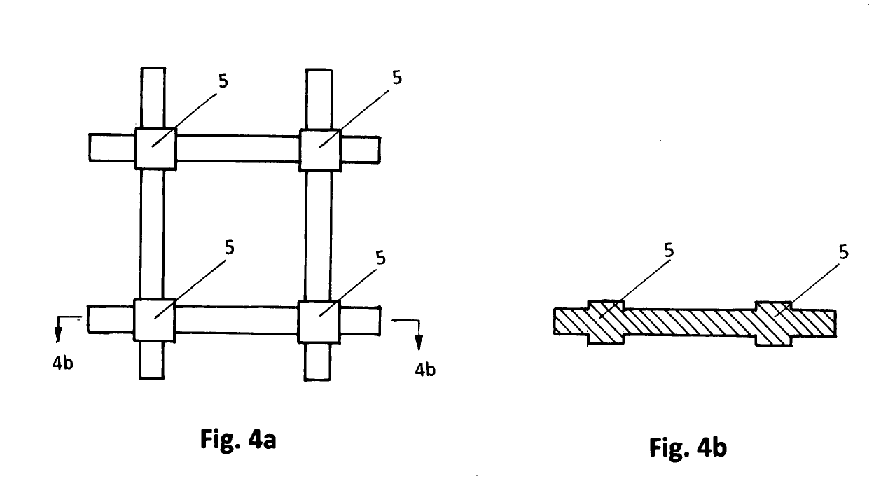

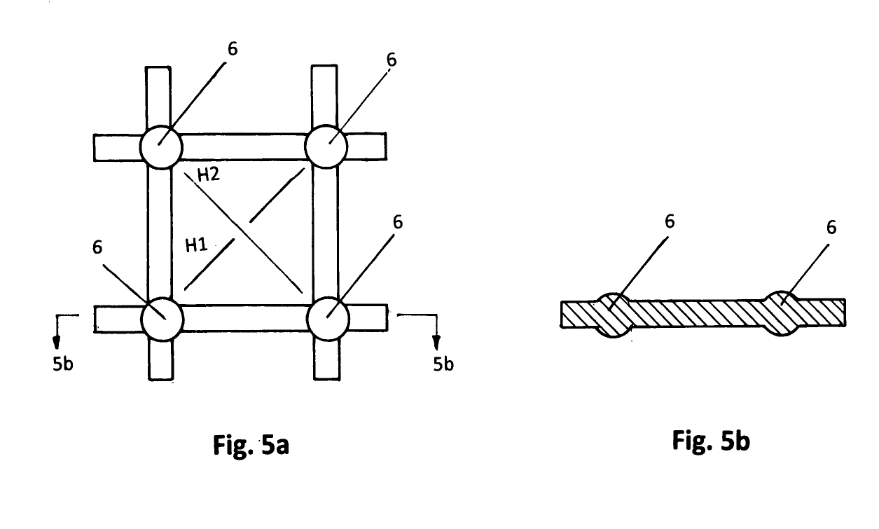

A cubical crossing configuration 5 of the crossing area is shown on FIG. 4a and a cross section is seen on FIG. 4b. A spherical crossing configuration 6 of the crossing areas of the threads is shown on FIG. 5a and FIG. 7, a cross section is demonstrated on FIG. 5b. The crossing configurations are rising above the surface of the rest of the tape. This creates more friction between the tape and the tissue, preventing rolling and dislodging of the tape after its insertion. The spherical crossing configuration 6 exerts less friction, then the cubical crossing configuration 5. The simple crossing configuration 3 has lesser friction.

Referring to embodiments on FIGS. 6a-6c and FIG. 7 are shown molded tapes having width of approximately 10-12 mm and a length between about 80 mm and 100 mm. The thickness of the thread is between 0.25 mm and 0.60 mm preferably about 0.30 mm. The diameter of the sphere of the spherical crossing configuration 6 is between 0.50 mm and 1.20 mm, preferably about 0.60 mm. The dimensions of the cube of the cubical crossing configuration 4 is between 0.50 mm×0.50 mm×0.50 mm and 1.20 mm×1.20 mm×1.20 mm, preferably about 0.60 mm×0.60 mm×0.60 mm. The macropores dimensions are preferably L1, about 1.80 mm and L2, about 1.80 mm, see FIG. 3a.

The molded tapes are made of only macropores having square shape, one of hypotenuse H1 is aligned with the longitudinal axis of the tape and the other hypotenuse H2 is at about 90° to the longitudinal axis of the tape, FIG. 5a. When the tape is stretched along hypotenuse H1, it becomes longer and hypotenouse H2 shorter, as a result the tape is elongated, after it is released, the tape goes back to the original length. This orientation of the macropores gives the tape a desired elasticity. The molded tapes shown on FIGS. 6b and 6c have a simple crossing configuration 3 of its three sections. The molded tape shown on FIG. 7 has simple crossing configurations 3 of the middle section and spherical crossing configurations 6 of the first end section 7 and the second end section 8. The edges of the tape, except the middle section, have a zigzag configuration 10. The edge is not sharp, which will not cut the tissue, but will insure a better grip. On FIG. 7 is shown a molded tape having three sections. The middle section has a simple crossing configuration 3, the first end section 7 the second end section 8 have spherical crossing configurations. Embodiment shown on FIG. 6c one of hypotenuse of the macropores of the middle section 9 are at about 45° to the longitudinal axis of the tape, so the middle section of the said tape can not be stretched along its longitudinal axis and across of it. As was mentioned above this is a very important feature of the middle section of the tape, because less movement of the part of the tape which lies over the urethra makes healing of the incision faster and reduces the risk of infection. A molded tape can be made with different combination of configuration of a middle and end sections. The length of the middle section 9 is between 10 mm and 30 mm, preferably about 20 mm. At the time of insertion of the tape under the urethra, the middle section 9 has to be placed over the urethra. After the surgery, unavoidable movement of the pelvic organs downward, including the urethra, stretches the tape. A restriction of the stretching of the middle section of the tape which lies over the urethra is very much desirable, because this allows faster healing of the incision of the vaginal wall which lies over the middle section of the tape preventing infection.

The very end 11 of the first and the second end sections are tapered, which will ease the insertion of the tape into the tissue.

Although embodiments of the present invention have been described herein with reference to the accompanying drawings, it is to be understood that the invention is not limited to those precise embodiments and various other changes and modifications may be affected herein by one skilled in the art without departing from the scope of the invention, as is limited only by the appended claims.

What is claimed is:

1. A method for treating female stress urinary incontinence comprising:
a) a tape made of woven mesh;
b) a tape having only macropores;
c) a macropore is constructed by single threads; and
d) at crossing areas of the threads, said threads are bonded together by heating said threads to the melting point of the surface of said threads.

2. A method of claim 1 wherein one of hypotenuse of macropores is aligned with the longitudinal axis of the tape.

3. A method for treating female stress urinary incontinence comprising:
a) a tape made of knitted mesh;
b) a tape having only macropores;
c) a macropore is constructed by single threads; and
d) at crossing areas of the threads, said threads are bonded together by heating said threads to the melting point of the surface of said threads.

4. A method of claim 3 wherein one of hypotenuse of macropores is aligned with the longitudinal axis of the tape.

5. A method for treating female stress urinary incontinence additionally comprising:
a) a molded tape;
b) a molded tape having only macropores;
c) a macropore is constructed by single threads; and
d) one of hypotenuse of macropores is aligned with the longitudinal axis of the molded tape.

6. A method of claim 5 wherein one of hypotenuse of macropores is at about 45° to the longitudinal axis of the molded tape.

7. A method for treating female stress urinary incontinence comprising:
a) a molded tape having three sections, a middle section, a first end section and a second end section; and
b) crossing areas of the threads having a simple crossing configuration, a spherical crossing configuration and a cubical crossing configuration.

8. A method of claim 7 wherein a middle section comprising:
a) having only macropores;
b) a macropore is constructed by single threads;
c) crossing areas of the threads having a simple crossing configuration;
d) one of hypotenuse of macropores is aligned with the longitudinal axis of said molded tape; and
e) a middle section is surrounded by a single thread.

9. A method of claim 8 wherein one of hypotenuse of macropores is at about 45° to the longitudinal axis of said molded tape.

10. A method of claim 7 wherein a first end section and s second end section comprising:
a) having only macropores;
b) a macropore is constructed by single treads;
c) one of hypotenuse of macropores is aligned with the longitudinal axis of the molded tape;
d) edges of the first end section and the second end section having zigzag configuration.

11. A method of claim 10 wherein crossing areas of the treads having a spherical crossing configuration.

12. A method of claim 10 wherein crossing areas of the threads having a cubical crossing configuration.
